(19) United States
(12) Patent Application Publication
(10) Pub. No.: US 2021/0363528 A1
(43) Pub. Date: Nov. 25, 2021

(54) BIOLOGICS ENGINEERING VIA APTAMOMIMETIC DISCOVERY

(71) Applicant: X Development LLC, Mountain View, CA (US)

(72) Inventors: Ivan Grubisic, Oakland, CA (US); Ray Nagatani, San Francisco, CA (US)

(21) Appl. No.: 16/877,729

(22) Filed: May 19, 2020

(51) Int. Cl. C12N 15/115; C12N 15/10; C40B 40/08; G16B 5/00

(52) U.S. Cl. CPC C12N 15/115; G16B 5/00; C40B 40/08; C12N 15/1068

(57) ABSTRACT

The present disclosure relates to a biologics development platform that derives biologics from aptamers found to bind to a target. Particularly, aspects of the present disclosure are directed to generating sequencing data and analysis data for each unique aptamer of an aptamer library that binds to a target within a monoclonal compartment, inferring aptamer sequences derived from the sequencing data and the analysis data, identifying interaction points between the aptamer sequences and epitopes of the target based on structure or sequence motifs of the aptamer sequences, modeling molecular dynamics of interactions between the aptamer sequences and the epitopes to identify characteristics of the interaction points as requirements or restraints for the interactions, and inferring one or more amino acid sequences based on the characteristics of the interaction points derived from the interactions between aptamer sequences and the epitopes.

### FIELD

The present disclosure relates to development of biologics (e.g., therapeutic proteins such as antibodies), and in particular to a biologics development platform that derives biologics from aptamers found to bind to a target.

### BACKGROUND

Technologies to generate replenishable sources of target-specific biologics have revolutionized biomedical research and the diagnosis and treatment of diseases. Hybridoma technology is a highly effective and well-established method to generate murine monoclonal antibodies, and is widely used to produce antibodies for a variety of applications, including therapeutic antibodies. More recently, in vitro methods have been developed to generate target-specific biologics (e.g., therapeutic proteins such as monoclonal antibodies). Most notably, the development of in vitro display technologies such as phage display, has enabled rapid isolation of target-specific biologics from large peptide libraries.

The power of phage display as a discovery tool stems from two basic features of the system: (1) the linkage of genotype and phenotype, and (2) the ability to build display libraries that range in size from 10⁶ to 10¹¹ distinct binding candidates (e.g., potential biologics) and select those that bind the target. The physical linkage between the displayed peptide or protein and the gene that encodes it facilitates characterization of the displayed peptide or protein following selection of phage with a desired binding property. Once display of a parent peptide or protein has been demonstrated, it is possible to build display libraries of 10⁶-10¹¹ variants from which peptides or proteins having a desired binding property can be selected. That is, the first set of peptides or proteins that bind to an antigen of interest can be subsequently diversified, retaining the sequence features that initially caused binding while discovering new peptides or proteins that may bind the antigen.

Conventionally, the success of in vitro peptide or protein generation depends largely upon the quality and the size of the peptide or protein library. In the case of phage and yeast display libraries (the two most widely used methods), the size of a library is determined by the efficiency of host cell transformation. On the other hand, a lot of different factors can influence the quality of a library. This is especially true for the synthetic peptide or protein libraries, which typically have their sequence diversity concentrated in the complementarity determining regions (CDRs) generated by random combinations of mono- or trinucleotide units. For a biologic molecule to be manufactured, purified, and stored in large a quantity for commercial purpose, molecular properties such as the level of expression, binding affinity and avidity, stability, and solubility need to be optimal, and the biologic optimization can be time- and cost-intensive if the parental peptide or protein has poor initial properties. Consequently, it is desirable to generate initial hit peptides or proteins that have desirable physicochemical and biological properties.

### SUMMARY

In some embodiments, a method is provided that comprises synthesizing an aptamer library from one or more single stranded DNA or RNA (ssDNA or ssRNA) libraries; generating sequencing data and analysis data for each unique aptamer of the aptamer library that binds to one or more targets within one or more monoclonal compartments; generating, by a first prediction model, one or more aptamer sequences derived from the sequencing data and the analysis data; identifying, by a second prediction model, interaction points between the one or more aptamer sequences and epitopes of the one or more targets based on structure or sequence motifs of the one or more aptamer sequences; modeling, by a molecular dynamics model, molecular dynamics of interactions between the one or more aptamer sequences and the epitopes of the one or more targets to incorporate a time dimension to the interaction points between the one or more aptamer sequences and the epitopes of the one or more targets, where the modeling of the molecular dynamics identifies characteristics of the interaction points as requirements or restraints for the interactions; and generating, by a third prediction model, one or more amino acid sequences based on the characteristics of the interaction points derived from the interactions between the one or more aptamer sequences and the epitopes of the one or more targets.

In some embodiments, the method further comprises: partitioning a plurality of aptamers within the aptamer library into the monoclonal compartments that combined establish the compartment-based capture system, where each monoclonal compartment comprises the unique aptamer from the plurality of aptamers; capturing, by the compartment-based capture system, the one or more targets, where the capturing comprises the one or more targets binding to the unique aptamer within the one or more monoclonal compartments; and separating the one or more monoclonal compartments of the compartment-based capture system that comprise the one or more targets bound to the unique aptamer from a remainder of monoclonal compartments of the compartment-based capture system that do not comprise the one or more targets bound to a unique aptamer.

In some embodiments, the method further comprises: synthesizing another aptamer library from the one or more aptamer sequences derived from the sequencing data and the analysis data; partitioning a plurality of derived aptamers within the another aptamer library into monoclonal compartments that combined establish another compartment-based capture system, where each monoclonal compartment comprises a unique derived aptamer from the plurality of derived aptamers; capturing, by the another compartment-based capture system, the one or more targets, where the capturing comprises the one or more targets binding to the unique derived aptamer sequence within one or more monoclonal compartments; separating the one or more monoclonal compartments of the another compartment-based capture system that comprise the one or more targets bound to the unique derived aptamer from a remainder of monoclonal compartments of the another compartment-based capture system that do not comprise the one or more targets bound to a unique derived aptamer; and in response to the separating, validating the unique derived aptamer from each of the one or more monoclonal compartments as an aptamer having a high binding affinity with the one or more targets, where the interaction points between the one or more aptamer sequences are derived from the sequencing data and the analysis data in response to the validation of the unique derived aptamer from each of the one or more monoclonal compartments as the aptamer having the high binding affinity with the one or more targets.

In some embodiments, the method further comprises: generating, by a fourth prediction model, the structure or the sequence motifs of the one or more aptamer sequences derived from the sequencing data and the analysis data, the structure is a secondary structure, a tertiary structure, or a combination thereof; and grouping the one or more aptamer sequences into sets of aptamer sequences based on commonality between the structure or the sequence motifs, where the interaction points between the one or more aptamer sequences and the epitopes of the one or more targets are identified for each set of aptamer sequences, molecular dynamics of the interactions is modeled for each set of aptamer sequences, and one or more amino acid sequences are generated from the interactions between the one or more aptamer sequences and the epitopes of the one or more targets within each set of aptamer sequences.

In some embodiments, the method further comprises: synthesizing peptides, proteins or peptidomimetics with the predicted one or more amino acid sequences and variants thereof; and identifying, using a display assay, one or more peptides, proteins or peptidomimetics capable of binding the one or more targets.

In some embodiments, the method further comprises synthesizing a biologic using the one or more peptides, proteins or peptidomimetics identified as being capable of binding the one or more targets.

In some embodiments, the method further comprises: receiving a query concerning the one or more peptides, proteins or peptidomimetics capable of binding to the one or more targets; acquiring the one or more aptamer sequences as potentially satisfying the query; acquiring the one or more amino acid sequences and variants thereof as potentially satisfying the query based on the one or more aptamer sequences; validating, by the display assay, the one or more peptides, proteins or peptidomimetics as substantially or completely satisfying the query; and upon validating the one or more peptides, proteins or peptidomimetics and in response to the query, providing the one or more peptides, proteins or peptidomimetics as a result to the query.

In some embodiments, the characteristics include one or more of: (i) structural conformations and states, (ii) chemical groups or moieties, (iii) electrostatic interactions, (iv) force fields, (v) torsions, and (vi) bond parameters.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure will be better understood in view of the following non-limiting figures, in which:

FIGS. 1A and 1B show block diagrams of a aptamer development platform according to various embodiments;

FIG. 2 shows a block diagram of a biologics development platform according to various embodiments;

FIG. 3 shows a machine-learning modeling system for developing aptamers and biologics in accordance with various embodiments:

FIG. 4 shows an exemplary flow for aptamer development in accordance with various embodiments;

FIG. 5 shows an exemplary flow for biologics development in accordance with various embodiments;

FIG. 6 shows an exemplary flow for providing results to a query in accordance with various embodiments; and

FIG. 7 shows an exemplary computing device in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

I. Introduction

Peptidomimetics are compounds whose essential elements (pharmacophore) mimic a natural peptide or protein in three-dimensional space and retain the ability to interact with the target and produce the same effect (e.g., a biological effect). Peptidomimetics may be designed to circumvent some of the problems associated with a natural ligand such as a peptide or protein: e.g., stability against proteolysis (duration of activity) and poor bioavailability. Certain other properties, such as receptor selectivity or potency, often can be substantially optimized. Thus, peptidomimetics have great potential in drug and biologics discovery. The process for identifying peptidomimetics typically begins by designing and/or optimizing the variable region of a peptide, protein, or peptidomimetic (e.g., the CDR3 loops of an antibody) through phage display. The peptide, protein, or peptidomimetic acquired from phage display mimicking the binding site on a template (e.g., the natural ligand) and binding to a target are defined as mimotopes.

However, despite recent success in generating replenishable sources of target-specific biologics from mimotopes and binding assays such as phage display, limitations still exist for these technologies. For example, the identification and design of mimotopes relies on prior knowledge of ligand-receptor interactions, and thus biologics generated from mimotopes will use similar characteristics to known binding sites or receptors of a target. The identification and design of peptidomimetics fails to take into consideration that there may be other binding sites or receptors available for a target (e.g., a more accessible binding site) that allow for interaction with the target to produce a same or different effect (e.g., block a signal). Moreover, binder (potential drug or biologics) assessment through display technologies such as phage display are limited to libraries of 10⁶-10¹¹ variants due to the transformation efficiency of the phage. Whereas, synthetically developed nucleic acid libraries typically include 10¹⁴-10²⁴ random oligonucleotide strands (aptamers).

To address these limitations and problems, a biologics development system is disclosed herein that derives biologics from aptamers found to bind to a target. The important aspect is that knowledge can be gained (e.g., knowledge about epitopes on the target, which may be known or unknown and functional significance of those epitopes) from an aptamer development platform and then that knowledge can be expanded on with a biologics development platform to find other molecules (e.g., monoclonal antibodies) that may bind to the same epitopes of a target in question. For instance in an exemplary embodiment, a developmental process may comprise generating, by the aptamer development platform, sequencing data and analysis data for each unique aptamer of an aptamer library that binds to a target within a monoclonal compartment, inferring, by the aptamer development platform, aptamer sequences derived from the sequencing data and the analysis data, identifying, by the biologics development platform, interaction points between the aptamer sequences and epitopes of the target based on structure or sequence motifs of the aptamer sequences, modeling, by the biologics development platform, molecular dynamics of interactions between the aptamer sequences and the epitopes to identify characteristics of the interaction points as requirements or restraints for the interactions, and inferring, by the biologics development platform, one or more amino acid sequences based on the characteristics of the interaction points derived from the interactions between the aptamer sequences and the epitopes.

In some instances, the developmental process for the biologics development system may further comprise synthesizing peptides, proteins or peptidomimetics with the predicted one or more amino acid sequences and variants thereof; and identifying, using a display assay, one or more peptides, proteins or peptidomimetics capable of binding the one or more targets. A biologic may be synthesized using the one or more peptides, proteins or peptidomimetics identified as being capable of binding the one or more targets.

It will be appreciated that techniques disclosed herein can be applied to assess other biological material rather than aptamers. For example, alternatively or additionally, the techniques described herein may be used to assess the interaction between any type of biologic material (e.g., a whole or part of an organism such as E. coli, or a biologic product that is produced from living organisms, contain components of living organisms, or derived from human, animal, or microorganisms by using biotechnology) and a target, and derive a other type of biologic material therefrom based on the assessment.

II. Aptamer Development Techniques

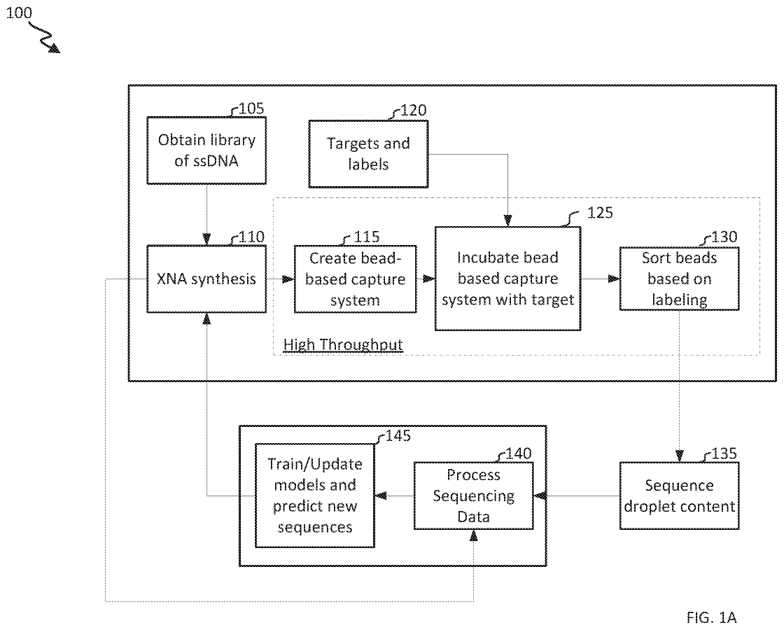

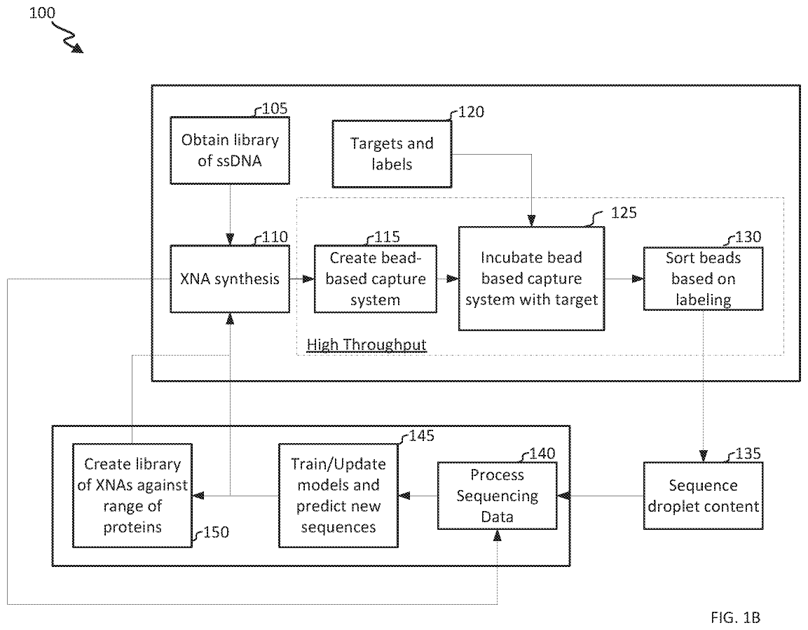

FIG. 1A shows a block diagram of an aptamer development platform 100 for strategically identifying particular aptamers for experiments to assess queries such as binding affinities or product inhibition with respect to one or more particular targets. In various embodiments, the aptamer development platform 100 implements screening-based techniques for aptamer discovery where each aptamer candidate sequence in a library is assessed based on the query (e.g., binding affinity with one or more targets or functionally capable of inhibiting one or more targets) in a high-throughput manner. In some embodiments, the aptamer development platform 100 implements machine learning based techniques for enhanced aptamer discovery where each aptamer candidate sequence in a library that satisfies the query is input into one or more machine-learning models to predict additional aptamer candidate sequences that potentially satisfy the query. In some embodiments, the aptamer development platform 100 further implements screening-based techniques for aptamer validation to validate or confirm that the predicted additional aptamer candidate sequences do satisfy the query (e.g., bind or inhibit the one or more targets). As should be understood, these techniques from screening through prediction to validation can be repeated in one or more closed loop processes sequentially or in parallel to ultimately assess any number of queries in a high through-put manner.

The aptamer development platform 100 includes obtaining one or more single stranded DNA or RNA (ssDNA or ssRNA) libraries at block 105. The one or more single stranded DNA or RNA (ssDNA or ssRNA) libraries may be obtained from a third party (e.g., an outside vendor) or may be synthesized in-house, and each of the one or more libraries typically contains up to 10¹⁷ different unique sequences. At block 110, the ssDNA or ssRNA of the one or more libraries are transcribed to synthesize a Xeno nucleic acid (XNA) aptamer library. XNA aptamer sequences such as threose nucleic acids (TNA) are synthetic nucleic acid analogues that have a different sugar backbone than the natural nucleic acids DNA and RNA. XNA may be selected for the aptamer sequences as these polymers are not readily recognized and degraded by nucleases, and thus are well-suited for in vivo applications. XNA aptamer sequences may be synthesized in vitro through enzymatic or chemical synthesis. For example, a XNA library of aptamers may be generated by primer extension of some or all of the oligonucleotide strands in a ssDNA library, flanking the aptamer sequences with fixed primer annealing sites for enzymatic amplification, and subsequent PCR amplification to create an XNA aptamer library that includes 10¹²-10¹⁷ aptamer sequences.

In some instances, the XNA aptamer library may be processed for application in downstream machine-learning processes. In certain instances, the aptamer sequences are processed for use as training data, test data, or validation data in one or more machine-learning models. In other instances, the aptamer sequences are processed for use as actual experimental data in one or more trained machine-learning models. In either instance, the aptamer sequences may be processed to generate initial sequence data comprising a representation of the sequence of each aptamer and optionally a count metric. The representation of the sequence can include one-hot encoding of each nucleotide in the sequence that maintains information about the order of the nucleotides in the aptamer. The representation of the sequence can additionally or alternatively include a string of category identifiers, with each category representing a particular nucleotide. The count metric can include a count of each aptamer in the XNA aptamer library.

At block 115, the aptamers within the XNA aptamer library are partitioned into monoclonal compartments (e.g., monoclonal beads or compartmentalized droplets) for high-throughput aptamer selection. For example, the aptamers may be attached to beads to generate a bead-based capture system for a target. Each bead may be attached to a unique aptamer sequence generating a library of monoclonal beads. The library of monoclonal beads may be generated by sequence-specific partitioning and covalent attachment of the sequences to the beads, which may be polystyrene, magnetic, glass beads, or the like. In some instances, the sequence-specific partitioning includes hybridization of XNA aptamers with capture oligonucleotides having an amine modified nucleotide for interaction with covalent attachment chemistries coated on the surface of a bead. In certain instances, the covalent attachment chemistries include N-hydroxysuccinimide (NHS) modified PEG, cyanuric chloride, isothiocyanate, nitrophenyl chloroformate, hydrazine, or any combination thereof.

At block 120, a target (e.g., proteins, protein complexes, peptides, carbohydrates, inorganic molecules, cells, etc.) is obtained. The target may be obtained as a result of a query posed by a user (e.g., a client or customer). For example, a user may pose a query concerning identification of ten aptamers with the highest binding affinity for a given target or twenty aptamers with the greatest ability to inhibit activity of a given target. In some instances, the target is tagged with a label such as a fluorescent probe. At block 125, the bead-based capture system is incubated with the labeled target to allow for the aptamers to bind with the target and form aptamer-target complexes.

At block 130, the beads having aptamer-target complexes are separated from the beads having non-binding aptamers using a separation protocol. In some instances, the separation protocol includes a fluorescence-activated cell sorting system (FACS) to separate the beads having the aptamer-target complexes from the beads having non-binding aptamers. For example, a suspension of the bead-based capture system may be entrained in the center of a narrow, rapidly flowing stream of liquid. The flow may be arranged so that there is separation between beads relative to their diameter. A vibrating mechanism causes the stream of beads to break into individual droplets (e.g., one bead per droplet). Before the stream breaks into droplets, the flow passes through a fluorescence measuring station where the fluorescent label which is part of the aptamer-target complexes is measured. An electrical charging ring may be placed at a point where the stream breaks into droplets. A charge may be placed on the ring based on the prior fluorescence measurement, and the opposite charge is trapped on the droplet as it breaks from the stream. The charged droplets may then fall through an electrostatic deflection system that diverts droplets into containers based upon their charge (e.g., droplets having beads with aptamer-target complexes go into one container and droplets having beads with non-binding aptamers go into a different container). In some instances, the charge is applied directly to the stream, and the droplet breaking off retains a charge of the same sign as the stream. The stream may then returned to neutral after the droplet breaks off

At block 135, the aptamers from the aptamer-target complexes are eluted from the beads and target, and amplified by enzymatic or chemical processes to optionally prepare for subsequent rounds of selection (repeat blocks 110-130, for example a SELEX protocol). The stringency of the elution conditions can be increased to identify the tightest-binding or highest affinity sequences. In some instances, once the aptamers are separated and amplified, the aptamers may be sequenced to identify the sequence and optionally a count for each aptamer. Optionally, the beads having non-binding aptamers are eluted from the beads, and amplified by enzymatic or chemical processes. In some instances, once the non-binding aptamers are separated and amplified, the non-binding aptamers may be sequenced to identify the sequence and optionally a count for each non-binding aptamer.

At block 140, a data set including the sequence, the count, and/or an analysis performed based on the separation protocol (e.g., a binary classifier or a multiclass classifier) for each aptamer that has gone through the selection process of steps 110-130 is processed for application in downstream machine-learning processes. The data set may include the sequence, the count, and/or the analysis from the binding aptamers (those that formed the aptamer-target complexes), the non-binding aptamers (those that did not form the aptamer-target complexes), or the combination thereof. In general, there are different types of binders (e.g., agonist, antagonist, allosteric, etc.) and those would be characteristics that the system may be configured to distinguish between the different types of binders during training, testing, and/or experimental analysis. In some instances, the sequence, count, and/or analysis for each aptamer is processed for use as training data, test data, or validation data in one or more machine-learning models. In other instances, the sequence, count, and/or analysis for each aptamer is processed for use as actual experimental data in one or more trained machine-learning models. In either instance, the sequence, count, and/or analysis for each aptamer may be processed to generate selection sequence data comprising a representation of the sequence of each aptamer, a count metric, an analysis metric, or any combination thereof. The representation of the sequence can include one-hot encoding of each nucleotide in the sequence that maintains information about the order of the nucleotides in the aptamer. The representation of the sequence can additionally or alternatively include other features concerning the sequence and/or aptamer, for example, post-translational modifications, binding sites, enzyme active sites, local secondary structure, kmers or characteristics identified for specific kmers, etc. The representation of the sequence can additionally or alternatively include a string of category identifiers, with each category representing a particular nucleotide. The count metric may include a count of the aptamer detected subsequent to an exposure to the target (e.g., during incubation and potentially in the presence of other aptamers). In some instances, the count metric includes a count of the aptamer detected subsequent to an exposure to the target in each round of selection. The analysis metric may include a binary classifier such as functionally inhibited the target, functionally did not inhibit the target, bound to the target, or did not bound to the target, a multiclass classifier such as a level of functional inhibition or a gradient scale for binding affinity.

At block 145, one or more machine-learning models are trained using the initial sequence data (from block 110), the selection sequence data (from block 135), or a combination thereof processed in block 140. The one or more machine-learning models may include a neural network, such as a feedforward neural network, recurrent neural network, convolutional neural network, and/or a deep neural network. The machine-learning models may be trained using training data, test data, and validation data based on sets of initial sequence data and selection sequence data to predict sequences for derived aptamers (e.g., aptamers not experimentally determined by a selection process but predicted based on aptamers experimentally determined by a selection process) and optional counts and/or analytics for the predicted sequences for derived aptamers. A loss function, such as a Mean Square Error (MSE), likelihood loss, or log loss (cross entropy loss), may be used to train each of the one or more machine-learning models. In some instances, a machine-learning model may be trained for predicting sequences for derived aptamers using the initial sequence data and/or the selection sequence data. Another machine-learning model may be trained for predicting binding counts for the predicted sequences for derived aptamers using the initial sequence data and/or the selection sequence data. Another machine-learning model may be trained for predicting analytics such as binding affinity for the predicted sequences for derived aptamers using the initial sequence data and/or the selection sequence data.

The trained machine-learning models can then be used to predict sequences for derived aptamers and optional counts and/or analytics for the predicted sequences for derived aptamers. For example, a subset of the aptamers experimentally determined by the selection process to satisfy the query (e.g., aptamers that have high binding affinity with a target or predicted counts due primarily to high binding affinity with a target) can be identified and separated from aptamers experimentally determined by the selection process to not satisfy the query. The subset of the aptamers experimentally determined by the selection process to satisfy the query can then be input into one or more machine learning models to identify in silico derived aptamer sequences (e.g., aptamer sequences that are derivatives of the experimentally selected aptamers) and optionally counts and analytics for the derived aptamer sequences. Optionally, the subset of the aptamers experimentally determined by the selection process to not satisfy the query can also be input into one or more machine learning models to assist in identifying in silico derived aptamer sequences (e.g., aptamer sequences that are derivatives of the experimentally selected aptamers) and optionally counts and analytics for the derived aptamer sequences.

The output can trigger experimental testing of some or all of the in silico derived aptamer sequences to experimentally measure analytics such as binding affinities with the target and/or binding affinities with one or more other targets. The experimental testing may be conditioned on input from a user. For example, a user device may present an interface in which the in silico derived aptamer sequences are identified along with input components configured to receive input to modify the in silico derived aptamer sequences (e.g., by removing or adding aptamers) and/or to generate an experiment-instruction communication to be sent to another device and/or other system. The experiment can include producing each of the in silico derived aptamer sequences. These aptamers can then be validated in the wet lab in either individual or bulk experiments. For example, the user can access a single aptamer (e.g. oligonucleotide). The single aptamer can be provided by an aptamer source, such as Twist Biosciences, Agilent, IDT, etc. The aptamer can be used to conduct biochemical assays (e.g. gel shift, surface plasma resonance, bio-layer interferometry, etc.). In some instances, multiple aptamers in a singular pool can be used to rerun the equivalent SELEX protocol (e.g., blocks 115-140) to identify enriched aptamers. Results can be assessed to determine whether the computational experiments are verified. In some instances, selections can be run in a digital format (i.e., ones that give a functional output per sequence) to validate particular sequences. The validated sequences can be used to update the training set because the pair of sequence and affinity metric can be both normalized and calibrated.

FIG. 1B shows a block diagram of an alternative aptamer development platform 100 for strategically identifying particular aptamers for experiments to assess queries such as binding affinities or product inhibition with respect to one or more particular targets. In various embodiments, the aptamer development platform 100 implements screening-based techniques for aptamer discovery where each aptamer candidate sequence in a library is assessed based on the query (e.g., binding affinity with one or more targets or functionally capable of inhibiting one or more targets) in a high-throughput manner, as described with respect to FIG. 1A. Additionally, the aptamer development platform 100 may implement machine learning based techniques for enhanced aptamer discovery where a library of predicted sequences for derived aptamers against a range of queries and/or targets is generated for subsequent processing (e.g., used as a base library of aptamer sequences in experimental testing (steps 110-140), instead of a random pool of oligonucleotides or aptamers, to answer a new query).

More specifically, at step 150, the output of the trained machine-learning models (sequences for derived aptamers and optional counts and/or analytics of the predicted sequences for derived aptamers) can trigger recording of some or all of the in silico derived aptamer sequences (e.g., positive and negative aptamer data such as predicted counts demonstrating increased binding affinity for a target or predicted counts demonstrating decreased binding affinity for a target) within a data structure (e.g., a database table). In some instances, the sequences for the derived aptamers are recorded in the data structure in association with additional information including the query, the one or more targets that are the focus of the query and basis for the genesis of the sequences for the derived aptamers, counts predicted for the sequences for the derived aptamers, analysis predicted for the sequences for the derived aptamers, or any combination thereof.

As should be understood, the aptamer development platform 100 described with respect to FIGS. 1A and 1B could be used for aptamer discovery where steps 110-140 are run in parallel to generate multiple monoclonal beads against multiple targets in association with one or more queries. Additionally or alternatively, the aptamer development platform 100 described with respect to FIGS. 1A and 1B could be used for aptamer discovery where steps 110-145 are run in parallel to generate multiple monoclonal beads against multiple targets in association with one or more queries and predict in parallel sequences for derived aptamers and optional counts and/or analytics for the predicted sequences for derived aptamers. The machine-learning models trained and used to make the predictions may be updated with results from the experiments and other machine-learning models using a distributed or collaborative learning approach such as federate learning which trains machine-learning models using decentralized data residing on end devices or systems. For example, a central or primary model may be updated or trained with results from all experiments being run and the results of the updating/training of the central or primary model may be propagated through to deployed secondary models (e.g., if information is obtained on cytokine a then the system may use that information to potential refine processes to identify for cytokine b).

III. Biologics Development Techniques

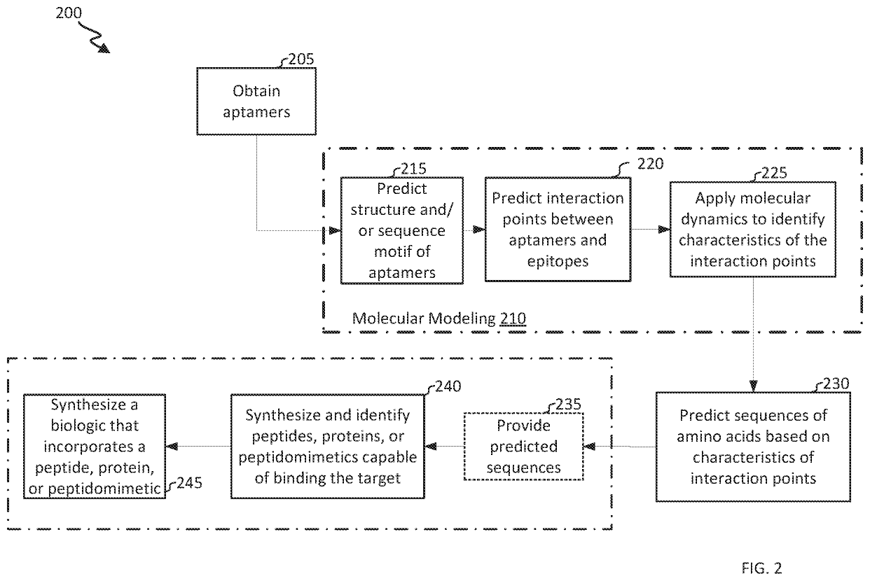

FIG. 2 shows a block diagram of a biologics development platform 200 for strategically identifying particular biologics for experiments to assess queries such as binding affinities or product inhibition with respect to one or more particular targets. In various embodiments, the biologics development platform 200 implements modeling-based techniques to identify sequences of aptamers binding to similar epitopes on the target, predict the structure of the aptamer sequences and likely interaction points between the aptamers and epitopes on the target required for the binding, identify characteristics of the interaction points as requirements or restraints for the interaction, and predict sequences of amino acids that can likely adopt a conformation to satisfy the requirements or restraints to make the same interactions with epitopes. In some instances, the biologics development platform 200 further implements synthesis and assay-based techniques to synthesize a peptide, protein or peptidomimetic with the predict sequences of amino acids and variants thereof, and use an in vitro screening technique (e.g., a display assay such as phage display) for identifying peptide(s), protein(s) or peptidomimetic(s) capable of binding the target. Thereafter, a biologic may be synthesized that incorporates one or more of the identified peptide(s), protein(s) or peptidomimetic(s) capable of binding the target. As used herein, a “biologic(s)”, also known as a biologic(al) medical product or biopharmaceutical, is any therapeutic product manufactured in, extracted from, or semi-synthesized from biological sources (e.g., a monoclonal antibody).

The biologics development platform 200 includes obtaining one or more aptamer libraries at block 205. The one or more aptamer libraries may be obtained from the aptamer development platform 100 as described with respect to FIGS. 1A and 1B. Each of the one or more aptamer libraries comprises some or all of the experimentally (in vitro) derived aptamers sequences and/or some or all of the in silico derived aptamer sequences. At block 210, molecular modeling is applied to interactions between aptamers from the one or more aptamer libraries and the target using structure prediction, docking prediction, epitope mapping, and/or molecular dynamics. For example, similar aptamers are going to bind to similar epitopes or portions of a target, and so molecular modeling may be used to predict structure of the similar aptamers, identify most likely interaction points of the similar aptamers to obtain a detailed mapping of potential interactions between aptamers and epitopes, and use molecular dynamics to incorporate a time dimension to structural and docking snapshots to better interpret the aptamer to epitope interactions.

At block 215, the structure and/or sequence motifs of aptamers from the one or more aptamer libraries is predicted, and the aptamers are grouped into sets based on similar structure and/or sequence motifs. A sequence motif is a nucleotide or amino-acid sequence pattern that is widespread and has, or is conjectured to have, a biological significance. The binding affinity and specificity of aptamers derive from their specific secondary and tertiary structures, which allow for the recognition of different target structures. The modeling of the secondary and tertiary structures takes into consideration the flexibility of the phosphodiester backbone and all possible base pairings, including noncanonical base pairing as well as the influence of hydrophobic interactions and best free energy conformations. In some instances, the secondary structure and/or sequence motifs of the aptamers is predicted. In other instances, the second structure, the tertiary structure, the sequence motifs, or a combination thereof is predicted for the aptamer.

Secondary structures occur as a result of intramolecular nucleotide pairing, may be predicted based on the nucleotide sequence, and are typically the reason for epitope-aptamer interactions. Among pseudoknots and G-quadruplex, the most common secondary structures for aptamers are stem-loops, which comprise four different substructures: (i) hairpin loop, (ii) bulge loop, (iii) interior loop, and (iv) multibranch loop, which can form more complex structures such as kissing hairpins. In some instances, the secondary structure is predicted by a computational model comprising one or more algorithms. The one or more algorithms may include: (i) Multiple EM for Motif Elicitation (MEME), Gapped local alignment of motifs (GLAM 2), Discriminative Regular Expression Motif Elicitation (DREME), or MEME-ChIP for discovering sequence motifs in a group of related DNA, RNA, XNA, or protein sequences, (ii) Multiple Em for Motif Elucidation in Rna's Including secondary Structures (MEMERIS) for searching sequence motifs in a set of RNA sequences and simultaneously integrating information about secondary structures, (iii) mfold or UNAfold for the prediction of the secondary structure of single stranded nucleic acids, and/or (iv) Aptamotif for the identification of sequence-structure motifs in SELEX-derived aptamers.

Two main approaches exist for the prediction of tertiary structures: (i) de novo modeling, which uses physics-based principles such as molecular dynamics or random sampling of the conformational landscape followed by screening with a statistical potential for scoring, and (ii) comparative modeling which uses related known structures as a template (e.g., homologous sequence structures from databases). In some instances, known structures are used in comparative modeling to infer the tertiary structure of the aptamers. In other instances, the tertiary structure is predicted by a computational model comprising one or more algorithms. The one or more algorithms may include: (i) a multi-scale, free energy landscape-based RNA folding model (e.g., a Vfold model), (ii) multi-scale molecular dynamics modeling approach (e.g., discrete molecular dynamics (DMD) simulations may be used to sample the vast conformational space of nucleotide molecules), (iii) stepwise assembly (SWA) for recursively constructing atomic-detail biomolecular structures, and/or (iv) model prediction via one or more of: RNAComposer, ModeRNA/SimRNA, Vfold, Rosetta, DMD, MC-Fold, 3dRNA, and AMBER, and data chemical-mapping methods such as SHAPE, DMS, CMCT, and mutate-and-map.

At block 220, the interaction points between the aptamers and epitopes are predicted. Since similar aptamers will bind to similar interaction points on a target, the interaction points between aptamers and epitopes may be predicted for each set of aptamers that is based on similar structure and/or sequence motifs. Interactions between aptamer and target are primarily based on polar and ionic interactions, in addition to shape complementarity that results in binding properties comparable to proteins such as monoclonal antibodies. In some instances, the interaction points may be predicted by a computational model comprising one or more docking algorithms and/or a biophysical approach towards epitope mapping. The one or more docking algorithms and/or biophysical approaches include: (i) GRAMM which utilizes rigid docking, six-dimensional shape complementarity, and fast Fourier transformation, (ii) FTDock which provides implementation of electrostatics and biochemical information, (iii) 3D-Dock which provides energy calculations, side chain optimization, and backbone refinement, (iv) Hex which is a spherical polar Fourier correlation method, (v) Gold, Autodock, or Autodock Vina which provides flexibility or rotamer-based search for both ligand and selected amino acids residues; docking in a determined binding pocket, energy-based scoring functions, and ability to handle surface pockets, (vi) PatchDock which utilizes local feature matching instead of six-dimensional transformation fitting for interaction prediction, (vii) Dot/Dot2 which utilize Poisson-Boltzmann methods for interaction predictions, (viii) ZDOCK or HDOCK which models docking between molecules using a template-based and template-free rigid docking mode, (ix) pepscan which utilizes a series of overlapping linear peptides that cover the entirety of the epitope and reacts arrays of the peptides with the aptamers and those segments that continue to bind represent a significant aspect of the epitope, (x) co-crystallization of the epitope:aptamer complex followed by solution of its atomic structure using x-ray diffraction and analysis, and/or (xii) nuclear magnetic resonance which provides a dynamic picture of the antigen:antibody complex in solution.

At block 225, molecular dynamics are used to incorporate a time dimension to the structural and docking snapshots to better interpret the aptamer to epitope interactions and identify characteristics of the interaction points as requirements or restraints for the interaction. Molecular dynamics simulations can describe nucleic acid and protein dynamics in detail, including the precise position of each atom at any instant in the simulation time along with the corresponding energies. For example, the molecular dynamics may start from the structural and docking snapshots obtained in blocks 215 and 220, which represents the atom coordinates of macromolecules. These molecules are immersed in silico in a solvent and have their positions updated along the simulation according to classical mechanic calculations of their interactions among themselves and with the solvent. The classical mechanic facet may be represented by empirical force fields with optimized parameters for biological molecules. Furthermore, quantitative analysis of the conformational ensembles of the molecules during the long-enough simulations can reveal the thermodynamic properties of the biological system.

The molecular dynamics simulations may be modeled, viewed, and analyzed using molecular modelling and visualization computer programs such as Visual Molecular Dynamics. The molecular modeling may be performed by a computational model comprising one or more algorithms processed on one or more graphic processing units (GPUs). In some instances, the one or more algorithms include: (i) AMBER or CHARMM for modeling force fields, specific torsions and bond's parameters, (ii) Particle mesh Ewald method (PME) for modeling electrostatic interactions, and/or (iii) coarse-grained models, normal mode analysis, or Markov state models for modeling force fields, specific torsions, bond parameters, and structural conformations and states. The structural and docking snapshots along with the molecular dynamics can identify characteristics of the interaction points as requirements or restraints for the interaction. In some instances, the characteristics include one or more of: (i) structural conformations and states, (ii) chemical groups or moieties, (iii) electrostatic interactions, (iv) force fields, (v) torsions, and (vi) bond parameters (e.g., parameters of covalent bonds between nucleotides).

At block 230, one or more sequences of amino acids are predicted that can likely adopt a conformation to satisfy the requirements or restraints to make the same or similar interaction(s) with the epitopes as the aptamers. The interactions between epitopes and peptides, proteins, or peptidomimetics are typically dependent upon a small number of contacts (e.g., residue to residue contacts) between the epitopes and peptide, proteins, or peptidomimetics. One approach for evaluating these contacts is to develop a full model of the complex using the requirements or restraints for the interaction(s). Another approach is to focus on specific, functionally relevant contacts to develop a partial model of the complex using the requirements or restraints for the interaction(s) rather than committing to a single model of the full complex. The full or partial model can then be used to evaluate whether predicted sequences of amino acids for the peptide, protein, or peptidomimetic template can accommodate the desired contacts while avoiding potential clashes with other parts of the target and to assess the overall stability of the complex. The peptide, protein, or peptidomimetic template can serve as a basis for a library of peptides, proteins, or peptidomimetics, e.g., with random mutations in the predicted sequences of amino acids to find a tighter binder. For example, if blocks 210-225 determine that a set of aptamers for an epitope have a high probability for a chemical moiety that is always in a same location creating a contact point between the aptamer and epitope, then a partial model may be generated to evaluate predicted sequences of amino acids that could achieve a same chemical moiety at a similar location in a CDR loop to create a similar point of contact between a peptide, protein, or peptidomimetic and the epitope. Essentially, the requirements or restraints for the interaction(s) define a search space for the amino acids and a library of peptides, proteins, or peptidomimetics that could satisfy the interaction constraints.

In some instances, at least a portion of the scaffold for the peptide, protein, or peptidomimetic template may be selected based on in silico docking potential peptides, proteins, or peptidomimetics to a model of the epitope, e.g., using a docking algorithm such as Hex or ZDOCK, as described herein with respect to block 220, which identifies a subset of potential peptides, proteins, or peptidomimetics as having preliminary complementarity to the epitope. The docking of the subset of the peptides, proteins, or peptidomimetics having preliminary complementarity to the epitope may be evaluated based on predicted contacts between the peptide, protein, or peptidomimetic and the epitope. In some instances, a prediction model such as random forest classifier, boosting and gradient descent, support vector machines and kernel methods, maximum entropy classifier, random ferns, and the like is used to statistically evaluate predicted contacts between the peptides, proteins, or peptidomimetics and epitopes. Based on the evaluation of the contacts, one or more sequences of amino acids are predicted that can likely adopt a conformation to satisfy the requirements or restraints to make the same interaction(s) with epitopes.

At optional block 235, the predicted sequences of amino acids are provided. For example, the predicted sequence of amino acids may be locally presented or transmitted to another device. The predicted sequences of amino acids may be output along with the query posed for the discovery or the set of aptamers determined to bind to the epitope. In some instances, the predicted sequences of amino acids are output to an end user or storage device. At block 240, synthesis techniques may be used to synthesize peptides, proteins or peptidomimetics with the predict sequences of amino acids and variants thereof, and an in vitro screening technique (e.g., a display assay such as phage display) may be used to identify peptide(s), protein(s) or peptidomimetic(s) capable of binding the target. For example, a library of peptides, proteins, or peptidomimetics may be computationally designed based on the predicted sequences of amino acids. The library may be designed to introduce variations (e.g., one or more amino acid substitutions) into the predicted sequences of amino acids to potentially improve binding with the epitope or target while not affecting expression or folding of the peptides, proteins, or peptidomimetics and/or functionality. At block 245, a biologic may be synthesized that incorporates one or more of the identified peptide(s), protein(s) or peptidomimetic(s) capable of binding the target.

It will be appreciated that techniques disclosed herein can be applied to assess other aptamers rather than XNA aptamers. For example, alternatively or additionally, the techniques described herein may be used to assess the interactions between any type of sequence of nucleic acids (e.g., DNA and RNA) and epitopes of a target. The important aspect is that knowledge can be gained (e.g., knowledge about epitopes on the target, which may be known or unknown and functional significance of those epitopes) from the aptamer development platform 100 (whether it utilizes XNA, DNA, RNA, or the like) and then that knowledge can be expanded on with the biologics development platform 200 to find other molecules (e.g., monoclonal antibodies) that may bind to the same epitopes target in question (e.g., in an instance where aptamers can't be used as the biologic).

IV. Modeling Techniques to Predict Sequences for Derived Aptamers and Amino Acid Sequences for Derived Peptides, Proteins or Peptidomimetics

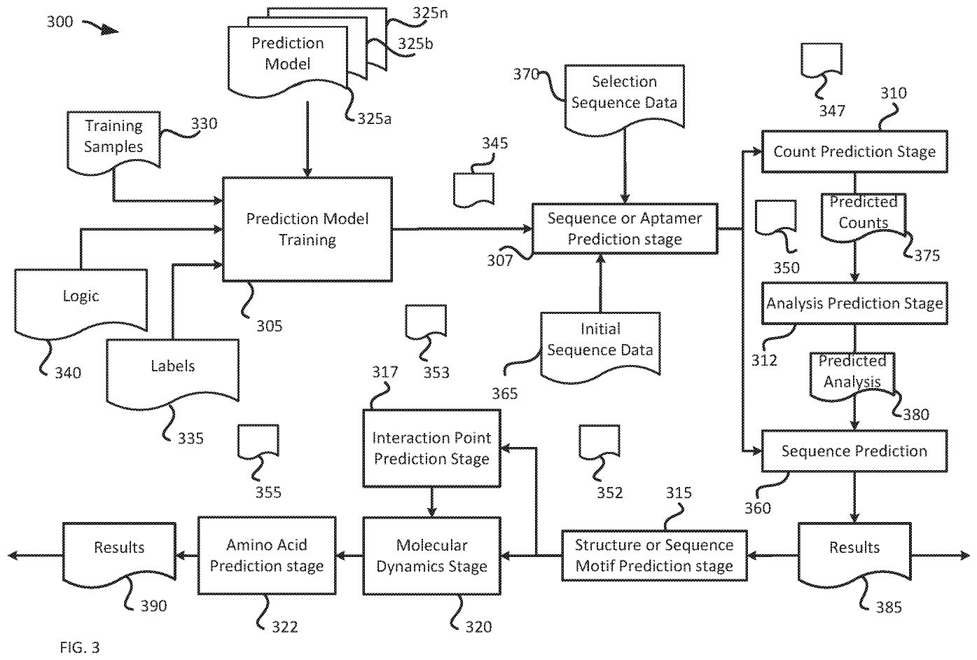

FIG. 3 shows a block diagram illustrating aspects of a machine-learning modeling system 300 for predicting sequences for derived aptamers and amino acid sequences for derived peptides, proteins, or peptidomimetics (e.g., aptamers, peptide, proteins, or peptidomimetics that answer a query posed by a user). As shown in FIG. 3, the predictions performed by the machine-learning modeling system 300 in this example include several stages: a prediction model training stage 305, a sequence or aptamer prediction stage 307, a count prediction stage 310, an analysis prediction stage 312, a structure or sequence motif prediction stage 315, an interaction point prediction stage 317, a molecular dynamics stage 320, and an amino acid prediction stage 322. The prediction model training stage 305 builds and trains one or more prediction models 325a-325n (‘n’ represents any natural number) to be used by the other stages (which may be referred to herein individually as a prediction model 325 or collectively as the prediction models 325). For example, the prediction models 325 can include a model for predicting sequences or aptamers not experimentally determined by a selection process but predicted based on aptamers experimentally determined by a selection process. The prediction models 325 can also include a model for predicting binding counts for the predicted sequences for derived aptamers. The prediction models 325 can also include a model for predicting analytics such as binding affinity for the predicted sequences for derived aptamers. The prediction models 325 can also include a model for predicting the structure or sequence motifs for derived aptamers. The prediction models 325 can also include a model for predicting interaction points between derived aptamers and epitopes. The prediction models 325 can also include a model for predicting the amino acids sequences for peptide, proteins, or peptidomimetics based on characteristics of the predicted interactions points. Still other types of prediction models may be implemented in other examples according to this disclosure.

A prediction model 325 can be a machine-learning model, such as a neural network, a convolutional neural network (“CNN”), e.g. an inception neural network, a residual neural network (“Resnet”) or NASNET provided by GOOGLE LLC from MOUNTAIN VIEW, CALIFORNIA, or a recurrent neural network, e.g., long short-term memory (“LSTM”) models or gated recurrent units (“GRUs”) models. A prediction model 325 can also be any other suitable machine-learning model trained to predict latent variables, sequence counts or aptamer sequences from experimentally determined aptamer sequences, structure or sequence motifs of derived aptamers, interaction points between aptamers and epitopes, and amino acid sequences for peptide, proteins, or peptidomimetics, such as a support vector machine, decision tree, coarse-grained models, normal mode analysis, or Markov state models, random forest classifier, boosting and gradient descent classifiers, a three-dimensional CNN (“3DCNN”), a dynamic time warping (“DTW”) technique, a hidden Markov model (“HMM”), etc., or combinations of one or more of such techniques—e.g., CNN-HMM or MCNN (Multi-Scale Convolutional Neural Network). In various instances, at least one of the prediction models 325a-n includes structures related to a loss function prior to training. The machine-learning modeling system 300 may employ the same type of prediction model or different types of prediction models for aptamer sequence prediction, aptamer count prediction, analysis prediction, structure or sequence motif prediction, interaction point prediction, and amino acid sequence prediction.

To train the various prediction models 325 in this example, training samples 330 for each prediction model 325 are obtained or generated. The training samples 330 for a specific prediction model 325 can include the initial sequence data, the selection sequence data, aptamer sequences, structure and sequence motifs, interaction points, and amino acid sequences, as described with respect to FIGS. 1A, 1B, and 2, and optional labels 335 corresponding to the initial sequence data, the selection sequence data, aptamer sequences, structure and sequence motifs, interaction points, and amino acid sequences. For example, for a prediction model 325 to be utilized to predict derived aptamer sequences based on a given sequence, the input can be the aptamer sequence itself or features extracted from the selection sequence data associated with the aptamer sequence and optional labels 335 can include known derivative sequences. Similarly, for a prediction model 325 to be utilized to predict a count or binding affinity for an aptamer sequence, the input can include the sequence and count features extracted from the initial sequence data and/or the selection sequence data associated with the sequence, and the optional labels 335 can include features indicating parameters for the count or binding affinity or a vector indicating probabilities for the count or binding affinity of the selection sequence data.

In some instances, the training process includes iterative operations to find a set of parameters for the prediction model 325 that minimizes a loss function for the prediction models 325. Each iteration can involve finding a set of parameters for the prediction model 325 so that the value of the loss function using the set of parameters is smaller than the value of the loss function using another set of parameters in a previous iteration. The loss function can be constructed to measure the difference between the outputs predicted using the prediction models 325 and the optional labels 335 contained in the training samples 330. Once the set of parameters are identified, the prediction model 325 has been trained and can be tested, validated, and/or utilized for prediction as designed.

In addition to the training samples 325, other auxiliary information can also be employed to refine the training process of the prediction models 325. For example, sequence logic 340 can be incorporated into the prediction model training stage 305 to ensure that the sequences or aptamers, counts, analysis, structures, sequence motifs, interaction points, molecular dynamics, and amino acids sequences predicted or modeled by a prediction model 325 do not violate the sequence, structural, or molecular dynamics logic 340. For example, binding affinity (the strength of the binding interaction between an aptamer and a target) is a characteristic that can drive aptamers to be present in greater numbers in a pool of aptamer-target complexes after a cycle of selection process. This relationship can be expressed in the sequence logic 340 such that as the binding affinity variable increases the predictive count increases (to represent this characteristic), as the binding affinity variable decreases the predictive count decreases. Moreover, an aptamer sequence generally has inherent logic among the different nucleotides. For example, GC content for an aptamer is typically not greater than 60%. This inherent logical relationship between GC content and aptamer sequences can be exploited to facilitate the aptamer sequence prediction.

According to some aspects of the disclosure presented herein, the logical relationship between the binding affinity and count can be formulated as one or more constraints to the optimization problem for training the prediction models 325. A training loss function that penalizes the violation of the constraints can be built so that the training can take into account the binding affinity and count constraints. Alternatively, or additionally, structures, such as a directed graph, that describe the current features and the temporal dependencies of the prediction output can be used to adjust or refine the features and predictions of the prediction models 325. In an example implementation, features may be extracted from the initial sequence data and combined with features from the selection sequence data as indicated in the directed graph. Features generated in this way can inherently incorporate the temporal, and thus the logical, relationship between the initial library and subsequent pools of aptamer sequences after cycles of the selection process. Accordingly, the prediction models 325 trained using these features can capture the logical relationships between sequence characteristics, selection cycles, aptamer sequences, and nucleotides.

Although the training mechanisms described herein mainly focus on training a prediction model 325, these training mechanisms can also be utilized to fine tune existing prediction models 325 trained from other datasets. For example, in some cases, a prediction model 325 might have been pre-trained using pre-existing aptamer sequence libraries. In those cases, the prediction models 325 can be retrained using the training samples 325 containing initial sequence data, experimentally derived selection sequence data, and other auxiliary information as discussed herein.

The prediction model training stage 305 outputs trained prediction models 325 including the trained sequence prediction models 345, trained count prediction models 347, trained analysis prediction models 350, trained structure or sequence motif prediction models 352, trained interaction point prediction models 353, and trained amino acid sequence prediction models 355. The trained sequence prediction models 345 may be used in the sequence prediction stage 307 to generate sequence predictions 360 for a subset or all of the initial sequence data 365 and/or the selection sequence data 370 identified during the experimental selection process (e.g., steps 110-140 described with respect to FIGS. 1A and 1B). The trained count prediction models 347 may be used in the count prediction stage 310 to generate count predictions 375 for the predicted sequences based on the initial sequence data 365 and/or the selection sequence data 370 identified during the experimental selection process (e.g., steps 110-140 described with respect to FIGS. 1A and 1B). The trained analysis prediction models 355 may be used in the analysis prediction stage 320 to generate analysis predictions 380 (e.g., a binary classifier such as binds to target or does not bind to target) for the predicted sequences based on the initial sequence data 365 and/or the selection sequence data 370 identified during the experimental selection process (e.g., steps 110-140 described with respect to FIGS. 1A and 1B). In some instances, a results stage 385 may use the sequence predictions 360, count predictions 375, analysis predictions 380, or any combination thereof to provide results to a query posed by a user. For example, the results stage 385, in response to query for top ten aptamers that bind a given target, may provide the sequence predictions for ten aptamers with the highest count or binding affinity for the given target.

The trained structure or sequence motif prediction models 352 may be used in the sequence prediction stage 315 (e.g., step 215 described with respect to FIG. 2) to generate structure or sequence motif predictions for a subset or all of the sequence predictions 360, the initial sequence data 365, and/or the selection sequence data 370 identified during the experimental and in silico selection process (e.g., steps 110-150 described with respect to FIGS. 1A and 1B). The trained interaction point prediction models 353 may be used in the interaction point prediction stage 317 (e.g., step 220 described with respect to FIG. 2) to generate interaction point predictions between the aptamers and epitopes predictions for a subset or all of the sequence predictions 360, the initial sequence data 365, and/or the selection sequence data 370 identified during the experimental and in silico selection process (e.g., steps 110-150 described with respect to FIGS. 1A and 1B). The structure or sequence motif predictions and the interaction point predictions may be input into the molecular dynamics stage 320 to incorporate a time dimension to the structural and docking snapshots to better interpret the aptamer to epitope interactions and identify characteristics of the interaction points as requirements or restraints for the interaction (e.g., step 225 described with respect to FIG. 2). The trained amino acid prediction models 355 may be used in the amino acid prediction stage 322 (e.g., step 230 described with respect to FIG. 2) to generate amino acid sequence predictions based on the characteristics of the interaction points from the molecular dynamics stage 320. In some instances, a results stage 390 may use the sequence predictions 360, count predictions 375, analysis predictions 380, amino acid sequence predictions, or any combination thereof to provide results to a query posed by a user. For example, the results stage 390, in response to query for top ten amino acid sequences that bind a given target, may provide the sequence predictions for ten amino acids with the highest count or binding affinity for the given target.

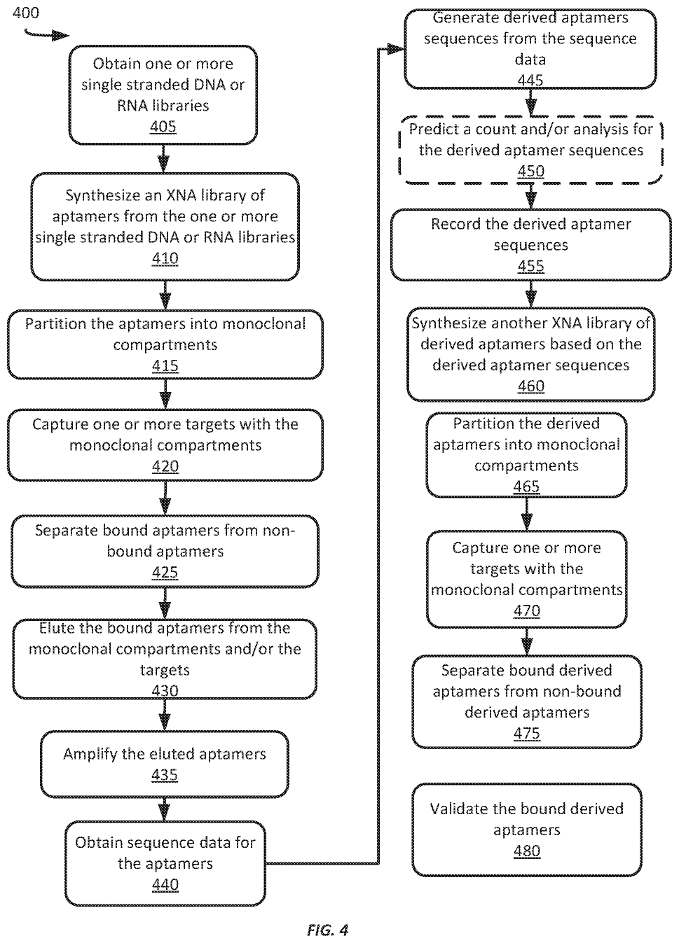

FIG. 4 is a simplified flowchart 400 illustrating an example of processing for developing aptamers using an aptamer development platform and a machine-learning modeling system and technique (e.g., the aptamer development platform 100 and machine-learning modeling system and technique 300 described with respect to FIGS. 1A, 1B, and 3). Process 400 begins at block 405, at which one or more single stranded DNA or RNA (ssDNA or ssRNA) libraries are obtained. The one or more ssDNA or ssRNA libraries comprise a plurality of ssDNA or ssRNA sequences. At optional block 410, an XNA aptamer library is synthesized from the one or more ssDNA or ssRNA libraries. The XNA aptamer sequences that make up the XNA aptamer library may be synthesized in vitro with a transcription assay that includes enzymatic or chemical synthesis. The XNA aptamer library comprises a plurality of aptamer sequences. It will be appreciated that techniques disclosed herein can be applied to assess other aptamers rather than XNA aptamers. For example, alternatively or additionally, the techniques described herein may be used to assess the interactions between any type of sequence of nucleic acids (e.g., DNA and RNA) and epitopes of a target. Thus, the following block may synthesize a DNA or RNA aptamer library as input for aptamer sequences rather than constructing an XNA library.

At block 415, the plurality of aptamers within the XNA aptamer library (optionally DNA or RNA libraries) are partitioned into monoclonal compartments that combined establish a compartment-based capture system. Each monoclonal compartment comprises a unique aptamer from the plurality of aptamers. In some instances, the one or more monoclonal compartments are one or more monoclonal beads. In some instances, each monoclonal compartment comprises a unique barcode (e.g., a unique sequence of nucleotides) for tracking identification of the compartment and/or the aptamer associated with the monoclonal compartment. At block 420, the compartment-based capture system is used to capture one or more targets. The capturing comprises the one or more targets binding to the unique aptamer within one or more monoclonal compartments. In some instances, the one or more targets are identified based on a query received from a user. As used herein, when an action is “based on” something, this means the action is based at least in part on at least a part of the something. At block 425, the one or more monoclonal compartments of the compartment-based capture system that comprise the one or more targets bound to the unique aptamer are separated from a remainder of monoclonal compartments of the compartment-based capture system that do not comprise the one or more targets bound to a unique aptamer. In some instances, the one or more monoclonal compartments are separated from the remainder of monoclonal compartments using a fluorescence-activated cell sorting system.

At block 430, the unique aptamer is eluted from each of the one or more monoclonal compartments and/or the one or more targets. At block 435, the unique aptamer from each of the one or more monoclonal compartments is amplified by enzymatic or chemical processes. At block 440, the unique aptamer from each of the one or more monoclonal compartments (e.g., the bound aptamers) are sequenced. The sequencing comprises using a sequencer to generate sequencing data and optionally analyze data for the unique aptamer from each of the one or more monoclonal compartments. The analysis data for the unique aptamer from each of the one or more monoclonal compartments may indicate the unique aptamer did bind to the one or more targets. In some instances, the sequencing further comprises generating count data for the unique aptamer from each of the one or more monoclonal compartments. In some instances, the sequencing further comprises sequences of unique aptamers from the remainder of the monoclonal compartments (e.g., non-bound aptamers). The sequencing further comprises using a sequencer to generate sequencing data and optionally analyze data for the unique aptamer from each of the remainder of the monoclonal compartments.

At block 445, one or more aptamer sequences are generated by a prediction model as being derived from the sequencing data and optionally the analysis data for at least some of the unique aptamers from the one or more monoclonal compartments. In some instances, the one or more aptamer sequences are generated as being derived from the sequencing data and optionally the analysis data and/or the count data for at least some of the unique aptamers from the one or more monoclonal compartments. Additionally, the one or more aptamer sequences may be generated as being derived from the sequencing data and optionally the analysis data for at least some of the unique aptamers from the remainder of the monoclonal compartments. Optionally at block 450, a count or analysis of the one or more aptamer sequences is predicted by another prediction model as being derived from the sequencing data and optionally the analysis data and/or count data for at least some of the unique aptamers from the one or more monoclonal compartment and/or at least some of the unique aptamers from the remainder of the monoclonal compartments. At block 455, the generated one or more aptamer sequences and optionally the predicted analysis data and/or count data are recorded in a data structure in association with the one or more targets.

At block 460, another XNA aptamer library (optionally a DNA or RNA library) is synthesized from the one or more aptamer sequences derived from the sequencing data and optionally the analysis data. At block 465, a plurality of derived aptamers within the another XNA aptamer library (optionally a DNA or RNA library) are partitioned into monoclonal compartments that combined establish another compartment-based capture system. Each monoclonal compartment comprises a unique derived aptamer from the plurality of derived aptamers. At block 470, another compartment-based capture system is used to capture the one or more targets. The capturing comprises the one or more targets binding to the unique derived aptamer sequence within one or more monoclonal compartments. At block 475, the one or more monoclonal compartments of the another compartment-based capture system that comprise the one or more targets bound to the unique derived aptamer are separated from a remainder of monoclonal compartments of the another compartment-based capture system that does not comprise the one or more targets bound to a unique derived aptamer. At block 480, in response to the separating, the unique derived aptamer from each of the one or more monoclonal compartments is validated as an aptamer having a high binding affinity with the one or more targets. As used herein, “binding affinity” is a measure of the strength of attraction between an aptamer and a target. As used herein, a “high binding affinity” is a result from stronger intermolecular forces between an aptamer and a target leading to a longer residence time at the binding site (higher “on” rate, lower “off” rate).

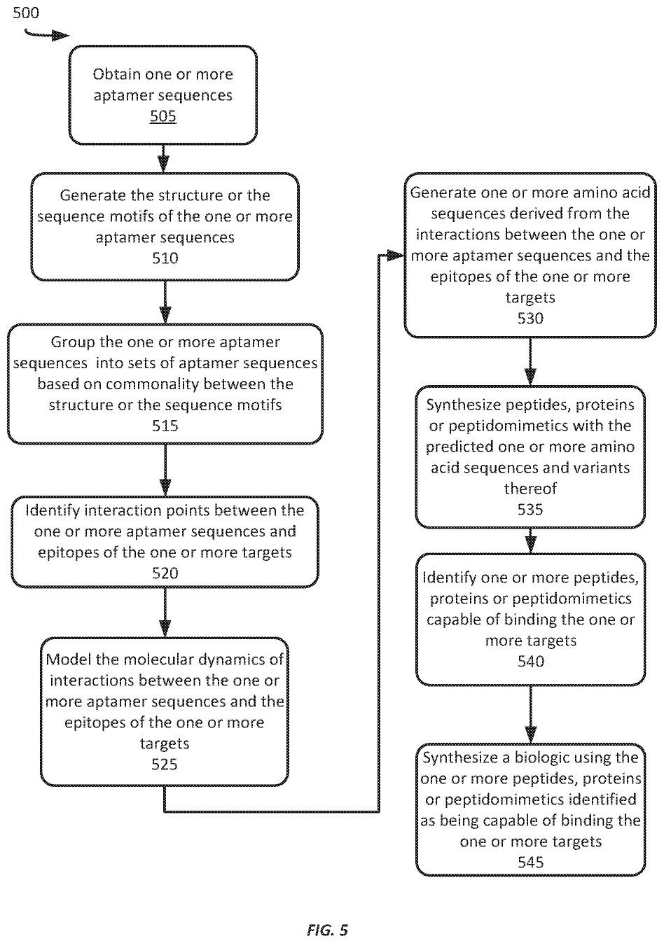

FIG. 5 is a simplified flowchart 500 illustrating an example of processing for developing biologics using a biologics development platform and a machine-learning modeling system and technique (e.g., the biologics development platform 200 and machine-learning modeling system and technique 300 described with respect to FIGS. 2 and 3). Process 500 begins at block 505, at which one or more aptamer sequences are obtained as being derived from the sequencing data and optionally analysis data for at least some unique aptamers. The sequencing data and analysis data may be generated for each unique aptamer of an aptamer library that binds to one or more targets within one or more monoclonal compartments, as described in detail with respect to flowchart 400 in FIG. 4. In some instances, the aptamer library is an XNA aptamer library. At block 510, the structure or the sequence motifs of the one or more aptamer sequences are generated (e.g., inferred or predicted using a prediction model). In some instances, the structure is a secondary structure, a tertiary structure, or a combination thereof. At block 515, the one or more aptamer sequences may be grouped into sets of aptamer sequences based on commonality between the structure or the sequence motifs.

At block 520, interaction points between the one or more aptamer sequences and epitopes of the one or more targets are identified (e.g., inferred or predicted using a prediction model) based on structure or sequence motifs of the one or more aptamer sequences. Since similar aptamers will bind to similar interaction points on a target, the interaction points between aptamers and epitopes may be identified for each set of aptamers that is based on similar structure and/or sequence motifs. At block 525, the molecular dynamics of interactions between the one or more aptamer sequences and the epitopes of the one or more targets are modeled to incorporate a time dimension to the interaction points between the one or more aptamer sequences and the epitopes of the one or more targets. The modeling of the molecular dynamics identifies characteristics of the interaction points as requirements or restraints for the interactions. In some instances, the characteristics include one or more of: (i) structural conformations and states, (ii) chemical groups or moieties, (iii) electrostatic interactions, (iv) force fields, (v) torsions, and (vi) bond parameters.

At block 530, one or more amino acid sequences are generated (e.g., inferred or predicted using a prediction model) based on the characteristics of the interaction points derived from the interactions between the one or more aptamer sequences and the epitopes of the one or more targets. In some instances, at least a portion of the scaffold (e.g., the one or more amino acid sequences) for the peptide, protein, or peptidomimetic template may be selected based on in silico docking potential peptides, proteins, or peptidomimetics to a model of the epitope, e.g., using a docking algorithm such as Hex or ZDOCK, as described herein with respect to FIG. 2, which identifies a subset of potential peptides, proteins, or peptidomimetics as having preliminary complementarity to the epitope. The docking of the portion of the scaffold (e.g., the one or more amino acid sequences) having preliminary complementarity to the epitope may be evaluated based on predicted contacts between the peptide, protein, or peptidomimetic and the epitope. One approach for evaluating these contacts is to develop a full model of the complex using the requirements or restraints for the interaction(s). Another approach is to focus on specific, functionally relevant contacts to develop a partial model of the complex using the requirements or restraints for the interaction(s) rather than committing to a single model of the full complex. The full or partial model can then be used to evaluate whether the predicted sequences of amino acids for the peptide, protein, or peptidomimetic template can accommodate the desired contacts while avoiding potential clashes with other parts of the target and to assess the overall stability of the complex.

At block 535, peptides, proteins or peptidomimetics may be synthesized with the predicted one or more amino acid sequences and variants thereof. At block 540, one or more peptides, proteins or peptidomimetics capable of binding the one or more targets may be identified using a display assay such as a phage display. At block 545, a biologic may be synthesized using the one or more peptides, proteins or peptidomimetics identified as being capable of binding the one or more targets.

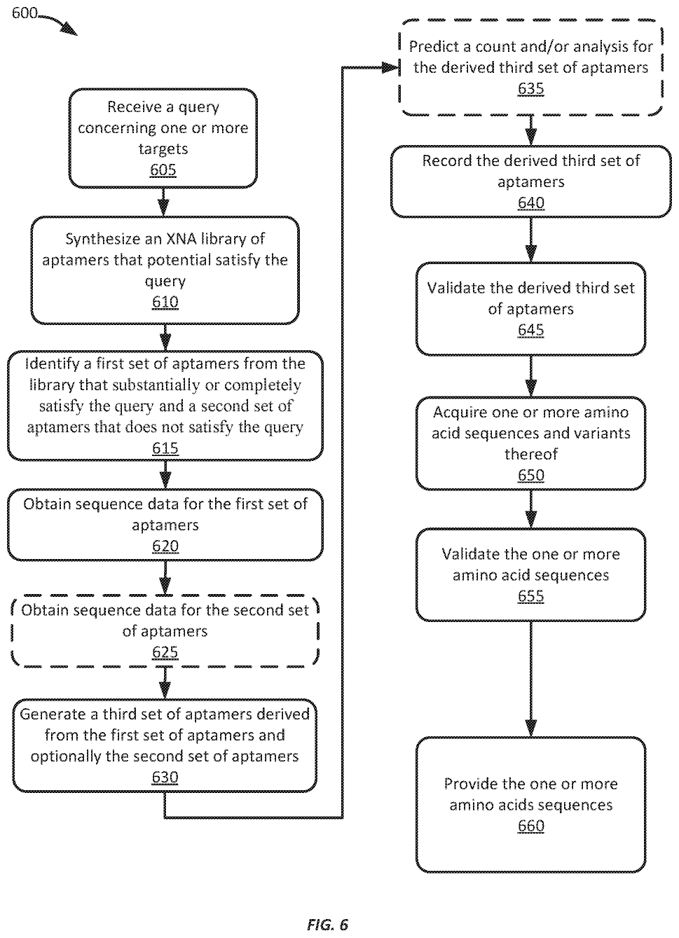

FIG. 6 is a simplified flowchart 600 illustrating an example of processing for providing results to a query using an aptamer development platform, a biologics development platform, and a machine-learning modeling system and technique (e.g., the aptamer development platform 100, the biologics development platform 200, and machine-learning modeling system and technique 300 described with respect to FIGS. 1A, 1B, 2 and 3). Process 600 begins at block 605, at which a query is received concerning one or more targets. For example, a user may pose a query concerning identification of ten amino acid sequences (or peptides, proteins or peptidomimetics comprising the amino acid sequences) with the highest binding affinity for a given target or twenty amino acid sequences (or peptides, proteins or peptidomimetics comprising the amino acid sequences) with the greatest ability to inhibit activity of a given target. At block 610, a library of aptamers that potentially satisfy the query is obtained. At block 615, a first set of aptamers from the library of aptamers is identified that substantially or completely satisfy the query and a second set of aptamers from the library of aptamers that does not substantially or completely satisfy the query. As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

At block 620, sequence data for the first set of aptamers is obtained. Optionally, analysis data and/or count data are also obtained for the first set of aptamers. In some instances, the analysis data for the first set of aptamers includes a binary classifier or a multiclass classifier selected based on the query. The binary classifier may indicate that each aptamer from the first set of aptamers functionally inhibited the one or more targets, functionally did not inhibit the one or more targets, bound to the one or more targets, or did not bound to the one or more targets; whereas the multiclass classifier may indicate a level of functional inhibition or a gradient scale for binding affinity with respect to each aptamer from the first set of aptamers and the one or more targets. At optional block 625, sequence data is obtained for the second set of aptamers.

At block 630, a third set of aptamers is generated by a prediction model as being derived from the sequence data for the first set of aptamers and optionally the analysis data for the first set of aptamers, the count data for the first set of aptamers, the second set of aptamers, or any combination thereof. At optional block 635, an analysis for each aptamer of the third set of aptamers is predicted by another prediction model as being derived from the sequence data for the first set of aptamers and the analysis data for the first set of aptamers. In some instances, the predicted analysis for the third set of aptamers includes the binary classifier or the multiclass classifier. At optional block 635, a count for each aptamer of the third set of aptamers is predicted by another prediction model as being derived from the sequence data for the first set of aptamers and the count data for the first set of aptamers.

At block 640, the third set of aptamers and optionally the predicted analysis and/or count for each aptamer of the third set of aptamers are recorded in a data structure in association with the one or more targets. At block 645, the third set of aptamers are validated as substantially or completely satisfying the query. At block 650, upon validating the third set of aptamers and in response to the query, one or more amino acid sequences and variants thereof are acquired as potentially satisfying the query based on the third set of aptamers. The one or more amino acid sequences and variants thereof are acquired as described in detail with respect to flowchart 500 and FIG. 5. At block 655, the amino acid sequences (or peptides, proteins or peptidomimetics comprising the amino acid sequences) are validated by a display assay as substantially or completely satisfying the query. The validation may include confirming that the amino acid sequences (or peptides, proteins or peptidomimetics comprising the amino acid sequences) do bind to the target. At block 660, upon validating the amino acid sequences (or peptides, proteins or peptidomimetics comprising the amino acid sequences) and in response to the query, providing the amino acid sequences (or peptides, proteins or peptidomimetics comprising the amino acid sequences) as a result to the query. In some instances, the providing may further comprise providing the third set of aptamers and optionally the first set of aptamers as a result to the query.

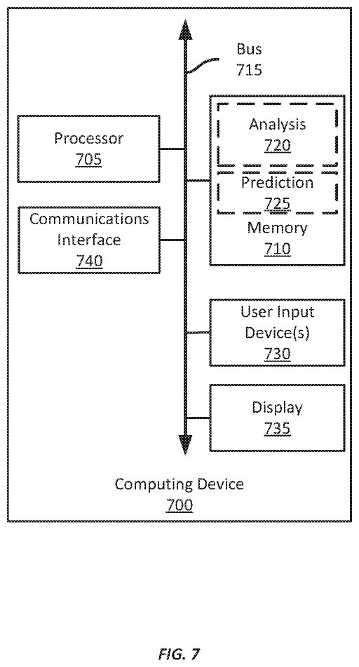

FIG. 7 illustrates an example computing device 700 suitable for use with systems and methods for developing aptamers and biologics or providing results to a query according to this disclosure. The example computing device 700 includes a processor 505 which is in communication with the memory 710 and other components of the computing device 700 using one or more communications buses 715. The processor 705 is configured to execute processor-executable instructions stored in the memory 710 to perform one or more methods for developing aptamers or biologics or providing results to a query according to different examples, such as part or all of the example method 400, 500, or 600 described above with respect to FIG. 4, 5, or 6. In this example, the memory 710 stores processor-executable instructions that provide sequence data analysis and amino acid sequence analysis 720 and aptamer and amino acid sequence prediction 725, as discussed above with respect to FIGS. 1A, 1B 2, 3, 4, 5, and 6.

The computing device 700, in this example, also includes one or more user input devices 730, such as a keyboard, mouse, touchscreen, microphone, etc., to accept user input. The computing device 700 also includes a display 735 to provide visual output to a user such as a user interface. The computing device 700 also includes a communications interface 740. In some examples, the communications interface 740 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

V. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method comprising:
synthesizing an aptamer library from one or more single stranded DNA or RNA (ssDNA or ssRNA) libraries;
generating sequencing data and analysis data for each unique aptamer of the aptamer library that binds to one or more targets within one or more monoclonal compartments;
generating, by a first prediction model, one or more aptamer sequences derived from the sequencing data and the analysis data;
identifying, by a second prediction model, interaction points between the one or more aptamer sequences and epitopes of the one or more targets based on structure or sequence motifs of the one or more aptamer sequences;
modeling, by a molecular dynamics model, molecular dynamics of interactions between the one or more aptamer sequences and the epitopes of the one or more targets to incorporate a time dimension to the interaction points between the one or more aptamer sequences and the epitopes of the one or more targets, wherein the modeling of the molecular dynamics identifies characteristics of the interaction points as requirements or restraints for the interactions; and
generating, by a third prediction model, one or more amino acid sequences based on the characteristics of the interaction points derived from the interactions between the one or more aptamer sequences and the epitopes of the one or more targets.

2. The method of claim 1, further comprising:
partitioning a plurality of aptamers within the aptamer library into the monoclonal compartments that combined establish the compartment-based capture system, wherein each monoclonal compartment comprises the unique aptamer from the plurality of aptamers;
capturing, by the compartment-based capture system, the one or more targets, wherein the capturing comprises the one or more targets binding to the unique aptamer within the one or more monoclonal compartments; and
separating the one or more monoclonal compartments of the compartment-based capture system that comprise the one or more targets bound to the unique aptamer from a remainder of monoclonal compartments of the compartment-based capture system that do not comprise the one or more targets bound to a unique aptamer.

3. The method of claim 2, further comprising:
synthesizing another aptamer library from the one or more aptamer sequences derived from the sequencing data and the analysis data;
partitioning a plurality of derived aptamers within the another aptamer library into monoclonal compartments that combined establish another compartment-based capture system, wherein each monoclonal compartment comprises a unique derived aptamer from the plurality of derived aptamers;
capturing, by the another compartment-based capture system, the one or more targets, wherein the capturing comprises the one or more targets binding to the unique derived aptamer sequence within one or more monoclonal compartments;
separating the one or more monoclonal compartments of the another compartment-based capture system that comprise the one or more targets bound to the unique derived aptamer from a remainder of monoclonal compartments of the another compartment-based capture system that do not comprise the one or more targets bound to a unique derived aptamer; and
in response to the separating, validating the unique derived aptamer from each of the one or more monoclonal compartments as an aptamer having a high binding affinity with the one or more targets,
wherein the interaction points between the one or more aptamer sequences are derived from the sequencing data and the analysis data in response to the validation of the unique derived aptamer from each of the one or more monoclonal compartments as the aptamer having the high binding affinity with the one or more targets.

4. The method of claim 3, further comprising:
generating, by a fourth prediction model, the structure or the sequence motifs of the one or more aptamer sequences derived from the sequencing data and the analysis data, the structure is a secondary structure, a tertiary structure, or a combination thereof; and
grouping the one or more aptamer sequences into sets of aptamer sequences based on commonality between the structure or the sequence motifs,
wherein the interaction points between the one or more aptamer sequences and the epitopes of the one or more targets are identified for each set of aptamer sequences, molecular dynamics of the interactions is modeled for each set of aptamer sequences, and one or more amino acid sequences are generated from the interactions between the one or more aptamer sequences and the epitopes of the one or more targets within each set of aptamer sequences.

5. The method of claim 4, further comprising:
synthesizing peptides, proteins or peptidomimetics with the predicted one or more amino acid sequences and variants thereof;
identifying, using a display assay, one or more peptides, proteins or peptidomimetics capable of binding the one or more targets; and
synthesizing a biologic using the one or more peptides, proteins or peptidomimetics identified as being capable of binding the one or more targets.

6. The method of claim 4, further comprising:
receiving a query concerning the one or more peptides, proteins or peptidomimetics capable of binding to the one or more targets;
acquiring the one or more aptamer sequences as potentially satisfying the query;
acquiring the one or more amino acid sequences and variants thereof as potentially satisfying the query based on the one or more aptamer sequences;
validating, by the display assay, the one or more peptides, proteins or peptidomimetics as substantially or completely satisfying the query; and
upon validating the one or more peptides, proteins or peptidomimetics and in response to the query, providing the one or more peptides, proteins or peptidomimetics as a result to the query.

7. The method of claim 1, wherein the characteristics include one or more of: (i) structural conformations and states, (ii) chemical groups or moieties, (iii) electrostatic interactions, (iv) force fields, (v) torsions, and (vi) bond parameters.

8. A computer-program product tangibly embodied in a non-transitory machine-readable storage medium, including instructions configured to cause one or more data processors to perform actions including:
obtaining an aptamer library from one or more single stranded DNA or RNA (ssDNA or ssRNA) libraries;
generating sequencing data and analysis data for each unique aptamer of the aptamer library that binds to one or more targets within one or more monoclonal compartments;
generating, by a first prediction model, one or more aptamer sequences derived from the sequencing data and the analysis data;
identifying, by a second prediction model, interaction points between the one or more aptamer sequences and epitopes of the one or more targets based on structure or sequence motifs of the one or more aptamer sequences;
modeling, by a molecular dynamics model, molecular dynamics of interactions between the one or more aptamer sequences and the epitopes of the one or more targets to incorporate a time dimension to the interaction points between the one or more aptamer sequences and the epitopes of the one or more targets, wherein the modeling of the molecular dynamics identifies characteristics of the interaction points as requirements or restraints for the interactions; and
generating, by a third prediction model, one or more amino acid sequences based on the characteristics of the interaction points derived from the interactions between the one or more aptamer sequences and the epitopes of the one or more targets.

9. The computer-program product of claim 8, wherein the actions further comprise:
obtaining another aptamer library from the one or more aptamer sequences derived from the sequencing data and the analysis data;
determining each unique aptamer of the another aptamer library that binds to the one or more targets within one or more monoclonal compartments;
in response to the determining, validating each unique aptamer from each of the one or more monoclonal compartments as an aptamer having a high binding affinity with the one or more targets,
wherein the interaction points between the one or more aptamer sequences are derived from the sequencing data and the analysis data in response to the validation of each unique aptamer from each of the one or more monoclonal compartments as the aptamer having the high binding affinity with the one or more targets.

10. The computer-program product of claim 9, wherein the actions further comprise:
generating, by a fourth prediction model, the structure or the sequence motifs of the one or more aptamer sequences derived from the sequencing data and the analysis data, the structure is a secondary structure, a tertiary structure, or a combination thereof; and
grouping the one or more aptamer sequences into sets of aptamer sequences based on commonality between the structure or the sequence motifs,
wherein the interaction points between the one or more aptamer sequences and the epitopes of the one or more targets are identified for each set of aptamer sequences, molecular dynamics of the interactions is modeled for each set of aptamer sequences, and one or more amino acid sequences are generated from the interactions between the one or more aptamer sequences and the epitopes of the one or more targets within each set of aptamer sequences.

11. The computer-program product of claim 10, wherein the actions further comprise:
obtaining synthesized peptides, proteins or peptidomimetics with the predicted one or more amino acid sequences and variants thereof; and
identifying, by a display assay, one or more peptides, proteins or peptidomimetics capable of binding the one or more targets.

12. The computer-program product of claim 11, wherein the aptamer library is an XNA aptamer library.

13. The computer-program product of claim 11, wherein the actions further comprise:
receiving a query concerning the one or more peptides, proteins or peptidomimetics capable of binding to the one or more targets;
acquiring the one or more aptamer sequences as potentially satisfying the query;
acquiring the one or more amino acid sequences and variants thereof as potentially satisfying the query based on the one or more aptamer sequences;
validating, by the display assay, the one or more peptides, proteins or peptidomimetics as substantially or completely satisfying the query; and
upon validating the one or more peptides, proteins or peptidomimetics and in response to the query, providing the one or more peptides, proteins or peptidomimetics as a result to the query.

14. The computer-program product of claim 8, wherein the characteristics include one or more of: (i) structural conformations and states, (ii) chemical groups or moieties, (iii) electrostatic interactions, (iv) force fields, (v) torsions, and (vi) bond parameters.

15. A system comprising:
one or more data processors; and
a non-transitory computer readable storage medium containing instructions which, when executed on the one or more data processors, cause the one or more data processors to perform actions including:
obtaining an aptamer library from one or more single stranded DNA or RNA (ssDNA or ssRNA) libraries;
generating sequencing data and analysis data for each unique aptamer of the aptamer library that binds to one or more targets within one or more monoclonal compartments;
generating, by a first prediction model, one or more aptamer sequences derived from the sequencing data and the analysis data;
identifying, by a second prediction model, interaction points between the one or more aptamer sequences and epitopes of the one or more targets based on structure or sequence motifs of the one or more aptamer sequences;
modeling, by a molecular dynamics model, molecular dynamics of interactions between the one or more aptamer sequences and the epitopes of the one or more targets to incorporate a time dimension to the interaction points between the one or more aptamer sequences and the epitopes of the one or more targets, wherein the modeling of the molecular dynamics identifies characteristics of the interaction points as requirements or restraints for the interactions; and
generating, by a third prediction model, one or more amino acid sequences based on the characteristics of the interaction points derived from the interactions between the one or more aptamer sequences and the epitopes of the one or more targets.

15. The system of claim 14, wherein the actions further comprise:
obtaining another aptamer library from the one or more aptamer sequences derived from the sequencing data and the analysis data;
determining each unique aptamer of the another aptamer library that binds to the one or more targets within one or more monoclonal compartments,
in response to the determining, validating each unique aptamer from each of the one or more monoclonal compartments as an aptamer having a high binding affinity with the one or more targets,
wherein the interaction points between the one or more aptamer sequences are derived from the sequencing data and the analysis data in response to the validation of each unique aptamer from each of the one or more monoclonal compartments as the aptamer having the high binding affinity with the one or more targets.

16. The system of claim 15, wherein the actions further comprise:
generating, by a fourth prediction model, the structure or the sequence motifs of the one or more aptamer sequences derived from the sequencing data and the analysis data, the structure is a secondary structure, a tertiary structure, or a combination thereof; and
grouping the one or more aptamer sequences into sets of aptamer sequences based on commonality between the structure or the sequence motifs,
wherein the interaction points between the one or more aptamer sequences and the epitopes of the one or more targets are identified for each set of aptamer sequences, molecular dynamics of the interactions is modeled for each set of aptamer sequences, and one or more amino acid sequences are generated from the interactions between the one or more aptamer sequences and the epitopes of the one or more targets within each set of aptamer sequences.

17. The system of claim 16, wherein the actions further comprise:
obtaining synthesized peptides, proteins or peptidomimetics with the predicted one or more amino acid sequences and variants thereof; and
identifying, by a display assay, one or more peptides, proteins or peptidomimetics capable of binding the one or more targets.

18. The system of claim 17, wherein the aptamer library is an XNA aptamer library.

19. The system of claim 17, wherein the actions further comprise:
receiving a query concerning the one or more peptides, proteins or peptidomimetics capable of binding to the one or more targets;
acquiring the one or more aptamer sequences as potentially satisfying the query;
acquiring the one or more amino acid sequences and variants thereof as potentially satisfying the query based on the one or more aptamer sequences;
validating, by the display assay, the one or more peptides, proteins or peptidomimetics as substantially or completely satisfying the query; and
upon validating the one or more peptides, proteins or peptidomimetics and in response to the query, providing the one or more peptides, proteins or peptidomimetics as a result to the query.

20. The system of claim 14, wherein the characteristics include one or more of: (i) structural conformations and states, (ii) chemical groups or moieties, (iii) electrostatic interactions, (iv) force fields, (v) torsions, and (vi) bond parameters.
